(19) United States
(12) Patent Application Publication
(10) Pub. No.: US 2006/0234369 A1
(43) Pub. Date: Oct. 19, 2006

(54) Implantable biosensor

(75) Inventor: Haris J. Sih, Minneapolis, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/269,384

(22) Filed: Nov. 8, 2005

Related U.S. Application Data
(60) Provisional application No. 60/671,140, filed on Apr. 14, 2005.

(51) Int. Cl. A61N 1/00; C12M 1/34; C12N 5/06

(52) U.S. Cl. 435/287.1; 435/325; 607/2

(57) ABSTRACT

The present invention provides implantable biosensors. The biosensors comprise tissue or cells which are electrically excitable or are capable of differentiating into electrically excitable cells, and which can be used to monitor the presence or level of a molecule in a physiological fluid. In one embodiment, the tissue or cells are coupled via an electrical interface to an electronic measuring device or an electronic amplifying device. The biosensors may be placed (inserted or implanted) in any animal including a mammal. The present invention also provides various methods which employ a biosensor of the invention.

### CROSS-REFERENCE TO RELATED APPLICATION

This application claims the benefit of U.S. Provisional Application No. 60/671,140, filed on Apr. 14, 2005, under 35 U.S.C. § 119(e), which is hereby incorporated by reference.

### TECHNICAL FIELD

This application relates generally to implantable biosensors and, more particularly, to devices and methods which employ genetically modified electrically active cells to detect physiological events.

### BACKGROUND

Determining serum levels of analytes such as signaling molecules (e.g., hormones) normally entails withdrawing a blood sample from the patient, and then analyzing the sample on the benchtop. Obviously, this approach has limitations regarding the frequency of measurement and the inconvenience and discomfort associated with periodic blood draws. Optimization of implantable biosensors in order to allow continuous analyte measurement would lead to better monitoring of several human disorders such as diabetes mellitus. For example, if patients with diabetes were able to continuously see a display of glucose concentration in blood or tissue, they could better avoid extremes of glycemia and reduce their risk for long term complications. However, fibrosis of the foreign body capsule that typically develops around the implanted sensors 3-4 weeks after implantation may reduce the influx of substrates such as glucose and oxygen (Ward and Troupe, ASAIO J., 45:555 (1999); Updike et al., Diabetes Care, 23:208 (2000); Gilligan et al., Diabetes Care, 17:884 (1994)).

One of the fundamental tasks required of implantable medical devices is accurate real-time determination of relevant functional physiological needs. For instance, a cardiac pacemaker must determine the pacing rate required to supply the body with adequate cardiac output. Biosensors that transduce biological actions or reactions into signals amenable to ready detection and/or processing are well suited for such monitoring (Pancrazio et al., Ann. Biomed. Eng., 27:697 (1999)). Nonetheless, typical in vivo biosensors only approximate physiological function via the measurement of surrogate signals and so may introduce a prime source of error in biological monitoring (Celiker et al., Pacing Clin. Electrophysiol., 21:2100 (1998); Moura et al., Pacing Clin. Electrophysiol., 10:89 (1987)).

An alternative approach is to use a biologically based system that can sense physiological signals directly, thereby avoiding the approximation errors associated with surrogate signal sensing. Recently, the development of such a tissue-based biosensor was reported in which the endogenous signaling pathways of excitable tissue was exploited to couple the detection of in vivo circulating physiological inputs to a functionally responsive electrical output (Christini et al., Am. J. Physiol. Heart Circ. Physiol., 280:H2004 (1999)). Specifically, the activity and regulation of remotely engrafted neonatal cardiac tissue in a murine model system was monitored. The chronotropic dynamics of the exogenous excitable cardiac allografts were highly correlated with the activity of the endogenous heart. Moreover, pharmacological studies in this model system showed that the transplanted allografts were regulated by circulating catecholamines.

What is needed is a biologically based biosensor to detect particular molecules, e.g., circulating molecules.

### SUMMARY

The present invention provides implantable biosensors. The biosensors of the invention include donor tissue or cells, optionally transgenic (genetically altered) donor tissue or cells, that are electrically excitable or are capable of differentiating into electrically excitable tissue or cells, such as cardiac, neural or skeletal muscle donor tissue or cells, and are capable of binding a particular physiological molecule, which binding in turn produces a biological signal that can be detected (monitored) and, in one embodiment, correlated with the presence and/or amount of one or more physiological molecules. In one embodiment, the donor tissue or cells are xenogeneic relative to the intended recipient mammal, e.g., human, mouse, rat, pig, rabbit, sheep, bovine, horse, dog or cat. Such a biosensor may be used to repeatedly and chronically track changes in soluble molecules found in physiological fluid, e.g., in the blood, of an animal, e.g., a mammal. The invention thus provides for automatic measure of circulating molecules by an implantable system, in contrast to the infrequent and inconvenient monitoring of various circulating molecules by benchtop analysis of blood samples. In one embodiment, the invention provides for automatic monitoring of circulating molecules with a system including donor tissue or cells electrically connected to an implantable device having pacing/sensing capabilities, which can provide information that may optimize cardiac pacing or defibrillation therapy in contrast to current implantable pacemakers and defibrillators that do not have the capacity to measure circulating molecule concentrations. Moreover, the information obtained from the device may be automatically recorded and changes in the concentration of circulating molecules may be monitored so as to allow physicians to better manage patient health.

In one embodiment, the biosensor includes transgenic donor tissue or cells. The transgenic donor tissue or cells include an expression cassette comprising a transcriptional regulatory element operably linked to an open reading frame encoding a gene product, e.g., a protein, which is capable of being associated with the cell membrane and binding a molecule (ligand) found in physiological fluid. In one embodiment, the transgenic donor tissue or cells are electrically excitable tissue or cells and the binding of one or more ligands to the gene product alters the amount and/or activity of one or more intracellular second messengers. The alteration in the amount and/or activity of one or more intracellular second messengers in turn modulates the electrical potential of the transgenic donor tissue or cells. For example, binding of ANP or BNP to their receptor alters the amount of the intracellular second messenger cGMP; binding of glucagon to its receptor alters the amount of the intracellular second messenger cAMP; binding of catecholamines, e.g., epinephrine, norepinephrine, or other adrenergic receptor ligands to an adrenergic receptor alters the amount of the intracellular second messenger inositol triphosphate; binding of a ligand to a muscarinic receptor alters the amount of, for instance, inositol triphosphate and/or diacylglycerol; binding of angiotensin II to its receptor alters, for instance, the amount of inositol triphosphate, diacylglycerol and/or cGMP; and binding of vasopressin to its receptor alters, for example, the amount of intracellular calcium. Thus, by expressing a particular gene product in the transgenic donor tissue or cells, which are electrically excitable or are capable of differentiating into electrically excitable tissue or cells, and then calibrating the corresponding changes in refractoriness and/or conduction of the electrically excitable donor tissue or cells, this system can be used to measure the presence and/or concentration of a variety of soluble molecules present in physiological fluid. In one embodiment, the refractoriness and/or conduction of the electrically excitable donor tissue or cells are measured by the effective refractory period (ERP) of the donor tissue or cells. During the ERP, no evoked response is detected following delivery of a pacing pulse to the donor tissue or cells.

In one embodiment, two or more expression cassettes are introduced to electrically donor cells or tissue that are or are capable of differentiating into excitable cells. For instance, one expression cassette encodes an ion channel protein such as a protein which forms a nonselective cation channel, e.g., a cGMP sensitive ion channel including those found in the rods and cones of the eye and cAMP/cGMP sensitive ion channel including those found in olfactory sensory neurons, chloride channels, and including an ion channel that is not directly linked to intracellular second messengers, e.g., a K⁺ channel. That expression cassette, after introduction to donor tissue cells, may result in donor tissue or cells that are more or less sensitive to membrane potential alterations, for instance, less sensitive to refractoriness. That is, the presence of additional recombinantly expressed cell membrane bound proteins, such as those forming ion channels, may shift baseline electrical properties of donor tissue or cells having that protein(s). In one embodiment, the donor tissue or cells express recombinant nonselective cation channels, e.g., cGMP sensitive ion channels or cAMP/cGMP sensitive ion channels, from an exogenously introduced expression cassette having a transcription regulatory element, which is optionally preferentially expressed in donor tissue or cells, for instance, a tissue or cell specific promoter and/or enhancer, linked to an open reading frame for the nonselective cation channel.

The invention thus provides a transgenic mammalian cell which is electrically excitable or is capable of differentiating into an electrically excitable cell. The transgenic cell is augmented with an expression cassette comprising a transcriptional regulatory element operably linked to an open reading frame encoding a protein which is capable of associating with the cell membrane and binding a molecule found in physiological fluid of a mammal. The binding alters the amount and/or activity of one or more intracellular second messenger molecules in the transgenic mammalian cell, which one or more intracellular second messenger molecules in turn are capable of modulating the activity of one or more ion channels (native ion channels, recombinantly expressed ion channels, or both). In one embodiment, the modulation of the activity of one or more ion channels alters action potential conduction and/or refractoriness in the transgenic mammalian cell. In one embodiment, the transgenic mammalian cell is a stem cell, a bone marrow cell or a cardiac cell. In one embodiment, the transgenic mammalian cell is prepared using a viral vector to deliver the expression cassette, e.g., a retroviral, lentiviral or adeno-associated virus vector. Also provided is a composition comprising a plurality of the transgenic mammalian cells of the invention. In one embodiment, the plurality of transgenic cells forms a two- or three-dimensional structure, e.g., a sheet of cells. In one embodiment, the plurality of cells is attached to and/or embedded in a biocompatible matrix (scaffold) prior to administration/implantation.

In one embodiment, a biosensor system of the invention includes an implantable electrical stimulator and evoked response sensor coupled to electrically excitable donor tissue or cells. In one embodiment, the system may include a lead or a portion thereof having electrically excitable donor tissue or cells applied thereto, which system may be implanted in a mammal such as in a blood vessel of the mammal. In one embodiment, the donor tissue or cells are implanted and/or embedded in or near muscle tissue, e.g., cardiac tissue. In one embodiment, prior to implantation, the donor tissue or cells are embedded in and/or attached to a biocompatible matrix. In one embodiment, a mammal having a biosensor system is administered an angiogenic agent, e.g., VEGF, to enhance blood vessel formation to the donor transgenic mammalian tissue or cells. In one embodiment, the transgenic mammalian tissue or cells are implanted near a region with a vascular supply such as in or near a blood vessel.

In one embodiment, an expression cassette encoding a cell membrane receptor is introduced into an adult stem cell derived culture of electrically excitable cells. Once these cells are transplanted in a mammal such that they are exposed to the vasculature of the mammal, or once the transplanted cells in the mammal become vascularized, the donor cells are sensitive to the blood concentration of the ligand for the receptor. Activation of cell membrane receptors after ligand binding then triggers an alteration in the amount of one or more intracellular second messenger molecules known to modulate membrane bound ion channels. Modulation of the ion channels leads to an alteration in action potential conduction and/or refractoriness of the transplanted cells. By electrically pacing and sensing the transplanted transgenic tissue or cell, action potential characteristics can be detected, e.g., quantified, and optionally correlated to physiological fluid levels of the ligand. In one embodiment, a biosensor of the invention is employed to automatically measure glucagon levels, e.g., in diabetic patients. For example, cultured cardiac cells are infected with recombinant virus encoding a glucagon receptor. Optionally, cells are identified and/or selected that overexpress the glucagon receptor. Prior to transplantation, the cells may be cultured so as to form a coating on a lead or another support having a two-or three-dimensional shape, or introduced to a biocompatible material. Donor tissue or cells may then be transplanted into a patient in a vascularized location. An implantable medical device including pulse generator and sensing circuitry is connected to the transplanted donor tissue or cells through one or more leads each having one or more electrodes placed in or on the transplanted donor tissue or cells. Alternatively, an implanted biosensor device includes donor tissue or cells, lead(s), and a device including pulse generator and sensing circuitry, which are implanted into a vascularized location in a mammal, for instance, an artery. Changes in blood glucagon levels lead to an increase in intracellular cAMP in the transplanted donor tissue or cells via G proteins and adenylate cyclase (AC). Increased levels of cAMP then alter ionic currents, e.g., L-type Ca²⁺, fast Na⁺, K⁺ or Cl⁻ currents, which in turn modify refractoriness and/or conduction in the transplanted tissue or cells, for example, by modifying the ERP. Simple pacing protocols can measure the changes in refractoriness and/or conduction, such as by measuring the changes in ERP, and those changes can be correlated to blood glucagon levels.

Examples of electrically excitable donor tissue or cells include but are not limited to endocrine tissue or cells, egg cells, muscle tissue or cells found in cardiac tissue, and neuronal tissue or cells.

Examples of physiological molecules to be detected include but are not limited to molecules found in physiological fluid such as blood, seminal fluid, cerebrospinal fluid, lymphatic fluid and the like, molecules including but not limited to glucagon, insulin, endocrine, paracrine or autocrine hormones, e.g., thyrotropin hormone, ANP, BNP, pathogens, drugs or toxins. In one embodiment, molecules to be detected in blood include glucagon, e.g., using donor tissue or cells which express a recombinant glucagon receptor, a natriuretic peptide, e.g., ANP, BNP, CNP, or DNP-like NP, using donor tissue or cells which express a recombinant natriuretic peptide receptor such as NPR-A, NPR-B or NPR-C, angiotensin II, e.g., using donor tissue or cells which express a recombinant angiotensin receptor, or vasopressin, e.g., using donor tissue or cells which express a vasopressin receptor. In one embodiment, the binding of the molecule to an appropriate receptor triggers an alteration in the amount and/or activity of one or more intracellular second messengers, for instance, the binding results in an increase in the amount and/or activity of one or more intracellular second messengers. Intracellular second messengers include but are not limited to calcium, which can enter cells by L-type (voltage dependent) Ca²⁺ channels, cAMP, the levels of which are controlled by adenylcyclase (AC) and phosphodiesterase (PDE) (AC is activated by activating G protein), cGMP, the levels of which are controlled by guanylcyclase (GC) and cGMP phosphodiesterases, inositol triphosphate (IP₃), which binds to Ca release channels and diacyl glycerol (DAG) (the receptors for IP₃ are linked to phospholipase C-B via G_{q} and elicit hydrolysis of phosphatidyl inositol biphosphate to IP₃ and DAG which activates protein kinase (PKC), prostaglandylinositol cyclic phosphate, phospholipase C, DAG, PKC, cyclic ADP ribose, and arachidonic acid (via activation of phospholipase A2). Those second messenger molecules may modulate one or more ion channels including ion channels normally found in cardiac cells, rod cells, cone cells, olfactory cells or other cells, e.g., nonselective cation channels, and including native ion channels, recombinantly expressed ion channels, or both. Second messengers molecules which modulate ion channel activity in cardiac cells include cAMP for L-type Ca²⁺ channel and delayed rectifier K⁺ channels (Kᵣ⁺), PKC for L-type Ca²⁺ channels and delayed rectifier K⁺ channels, and cGMP for L-type Ca²⁺ channels.

In accordance with the invention, a biosensor which includes electrically excitable mammalian donor tissue or cells, is implanted in an animal. Preferably, the animal is a mammal including but are not limited to a human, mouse, rat, rabbit, ovine, canine, feline, bovine, equine, porcine, or caprine. In one embodiment, the biosensor includes donor tissue or cells that are autologous. In another embodiment, the biosensor includes donor tissue or cells that are xenogeneic. Prior to, after, or during transplantation, the donor tissue or cells may be electrically coupled to an implantable medical device, e.g., a pulse generator.

The invention also provides a system. The system includes mammalian donor tissue or cells which are electrically excitable or are capable of differentiating into electrically excitable cells which express a protein which is capable of associating with the cell membrane and binding a molecule found in physiological fluid of a mammal, which binding alters the amount and/or activity of one or more intracellular second messenger molecules which one or more intracellular second messenger molecules in turn modulate the activity of one or more ion channels; and an implantable medical device including an event detector electrically coupled to the mammalian tissue or cells and adapted to detect a modulation in conduction and/or refractoriness in the mammalian tissue or cells, and an implant controller coupled to the event detector, the implant controller adapted to produce a signal in response to a modulation in the conduction and/or refractoriness of the mammalian tissue or cells. In one embodiment, the implantable medical device is an implantable pulse generator including a pacing circuit. In one embodiment, the system includes pacing/sensing lead(s) each having electrode(s) placed in or on the mammalian donor tissue or cells. In one embodiment, the mammalian donor tissue or cells are transgenic mammalian tissue or cells, e.g., stem cells, bone marrow cells, or cardiac cells, augmented with an expression cassette comprising a transcriptional regulatory element, for instance, a promoter which is expressed in muscle cells operably linked to an open reading frame encoding a protein which is capable of associating with the cell membrane and binding a molecule found in physiological fluid of a mammal. The binding alters the amount and/or activity of one or more intracellular second messenger molecules which one or more intracellular second messenger molecules in turn modulate the activity of one or more ion channels. In one embodiment, the intracellular second messenger molecule is inositol triphosphate, diacylglycerol, calcium, cAMP and/or cGMP. In one embodiment, the one or more ion channels that are modulated include those associated with L-type Ca²⁺ current, Kᵣ⁺ current, fast Na⁺ current and/or Cl⁻ current. In one embodiment, the protein which binds the physiological molecule is a glucagon receptor, a receptor for a natriuretic peptide, e.g., ANP or BNP, an ion channel protein which binds ATP, an adrenergic receptor, a muscarinic receptor, an angiotensin receptor, or a vasopressin receptor. In another embodiment, the transgenic cells further comprise a second expression cassette comprising a second transcriptional regulatory element operably linked to an open reading frame encoding an ion channel protein, e.g., a chloride channel protein, a protein in an ion channel that is sensitive to cAMP or cGMP, or a nonselective cation channel protein.

In one embodiment, the mammalian donor tissue or cells are coupled via an electrical interface to an electronic measuring device or an electronic amplifying device. In another embodiment, the mammalian donor tissue or cells are coupled to endogenous tissue or cells in a mammal such as coupling to a blood vessel. In one embodiment, the mammalian donor tissue or cells are implanted in a region with a vascular supply or alternatively implanted in a region which can be vascularized, e.g., by administering an angiogenic agent (angiogenic growth factor). Preferably, prior to implantation, the donor tissue or cells are electrically excitable tissue or cells.

Also provided is a method for monitoring physiological function. The method includes introducing into a mammal, donor tissue or cells capable of carrying out a physiological function within the mammal, e.g., binding a molecule found in physiological fluid, which binding alters the amount and/or activity of one or more intracellular second messengers, the alteration of which in turn modifies the activity of one or more ion channels, thereby altering conductance and/or refractoriness of the donor tissue or cells. Prior to implantation, the donor tissue or cells may be introduced to a matrix, e.g., a biocompatible biodegradable or biocompatible nonbiodegradable matrix, or to an implantable device, e.g., a lead which is coated with donor tissue or cells. The donor tissue or cells may also be coupled via an electrical interface to an electronic measuring device. Once implanted, the donor tissue or cells may also be coupled to endogenous tissue or cells, including a blood vessel. The donor tissue or cells may also be electrically coupled to an implantable medical device, which in response to an alteration in the electrical potential of the donor tissue or cells, may deliver electrical stimuli. In one embodiment, an implantable biosensor includes donor tissue or cells, one or more leads and/or a pulse generator with sensing circuitry, and/or a delivery device which delivers a protein, glycoprotein, nucleic acid, or a drug, e.g., an angiogenic drug.

Thus, the invention provides a method to detect the presence or amount of a molecule in physiological fluid in a mammal. The method includes transmitting to an external system from a device implanted in the mammal, a signal corresponding to the presence and/or amount of one or more detected physiological molecules. The implanted device includes transgenic electrically excitable mammalian tissue or cells coupled to an event detector adapted to detect a modulation in conductance and/or refractoriness of the transgenic electrically excitable mammalian tissue or cells, and a controller coupled to the event detector and adapted to produce a signal in response to a modulation in the conductance and/or refractoriness in the transgenic electrically excitable mammalian tissue or cells. The transgenic electrically excitable mammalian tissue or cells are augmented with an expression cassette comprising a transcriptional regulatory element operably linked to an open reading frame encoding a protein which is capable of associating with the cell membrane and binding the one or more physiological molecules, which binding alters the amount and/or activity of one or more intracellular second messenger molecules in the transgenic electrically excitable mammalian cells and which one or more intracellular second messenger molecules in turn modulate the activity of one or more ion channels, which modulation is detected by the event detector. In one embodiment, an electrical signal is transmitted in response to modulation in the conduction and/or refractoriness of the transgenic electrically excitable mammalian tissue or cells. In one embodiment, the molecule to be detected is glucagon, a natriuretic peptide, e.g., ANP or BNP, vasopressin, angiotensin, or ATP. In one embodiment, the device is a tube, tubing, catheter, stent, wire, defibrillator, implantable drug infusion pump, wire leads, or a pacemaker.

Also provided is a method for monitoring the presence or amount of a molecule. The method includes monitoring the presence and/or amount of a molecule found in physiological fluid of a mammal, e.g., a human, implanted with a device comprising transgenic electrically excitable mammalian tissue or cells coupled to an event detector adapted to detect a modulation in conductance and/or refractoriness of the transgenic electrically excitable mammalian tissue or cells. The transgenic electrically excitable mammalian tissue or cells are augmented with an expression cassette comprising a transcriptional regulatory element operably linked to an open reading frame encoding a protein which is capable of associating with the cell membrane and binding the molecule, which binding alters the amount and/or activity of one or more intracellular second messenger molecules in the transgenic electrically excitable mammalian cell and which one or more intracellular second messenger molecules in turn modulate one or more ion channels, which modulation is detected by the event detector. In one embodiment, the transgenic electrically excitable mammalian tissue or cells are mammalian cardiac tissue or cells that are optionally incorporated in a biocompatible matrix. In one embodiment, the molecule which is monitored is glucagon, a natriuretic peptide, vasopressin, angiotensin, or ATP. In one embodiment, the device is a tube, tubing, catheter, stent, wire, defibrillator, implantable drug infusion pump, wire leads, or a pacemaker.

In still another aspect of the invention, there is provided a system which includes donor tissue or cells capable of carrying out a physiological function which elicits a detectable signal, e.g., an electrical signal including an alteration in conductance and/or refractoriness of the donor tissue or cells, and an electrical connection placed between the donor tissue or cells and an implantable medical device. If desired, an amplifier may be added to the system in order to boost the signal from the donor tissue or cells. In another embodiment, the donor tissue or cells may also be connected to a pacing circuit.

### BRIEF DESCRIPTION OF THE DRAWINGS

The drawings, which are not necessarily drawn to scale, illustrate generally, by way of example, but not by way of limitation, various embodiments discussed in the present document.

FIG. 1 is an illustration of an exemplary embodiment of a cardiac rhythm management (CRM) system including a biosensor and portions of the environment in which the CRM system operates.

FIG. 2 is a block diagram illustrating an exemplary embodiment of portions of the circuit of the CRM system.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their equivalents.

It should be noted that references to “an”, “one”, or “various” embodiments in this disclosure are not necessarily to the same embodiment, and such references contemplate more than one embodiment.

Definitions

By “muscle cell” or “muscle tissue” is meant a cell or group of cells derived from muscle, including, but not limited to, cells and tissue derived from skeletal muscle and cardiac muscle, and in some embodiments includes smooth muscle cells. The term includes muscle cells both in vitro and in vivo. Thus, for example, an isolated cardiomyocyte would constitute a “muscle cell” for purposes of the present invention, as would a muscle cell as it exists in muscle tissue present in a subject in vivo. The term also encompasses both differentiated and nondifferentiated muscle cells, such as myocytes, myotubes, myoblasts, both dividing and differentiated, cardiomyocytes and cardiomyoblasts.

By “cardiac cell” is meant a differentiated cardiac cell (e.g., a cardiomyocyte) or a cell committed to differentiating to a cardiac cell (e.g., a cardiomyoblast or a cardiomyogenic cell).

A “myocyte” is a muscle cell that contains myosin.

A “cardiomyocyte” is any cell in the cardiac myocyte lineage that shows at least one phenotypic characteristic of a cardiac muscle cell. Such phenotypic characteristics can include expression of cardiac proteins, such as cardiac sarcomeric or myofibrillar proteins or atrial natriuretic factor (ANP), or electrophysiological characteristics. Cardiac sarcomeric or myofibrillar proteins include, for example, atrial myosin heavy chain, cardiac-specific ventricular myosin heavy chain, desmin, N-cadherin, sarcomeric actin, cardiac troponin I, myosin heavy chain, and Na/K ATPase. Electrophysiological characteristics of a cardiomyocyte include, for example, Na⁺ or K⁺ channel currents. Similarly, by “skeletal muscle cell” is meant any cell in the skeletal muscle cell lineage that shows at least one phenotypic characteristic of a skeletal muscle cell. Such phenotypic characteristics can include expression of skeletal muscle proteins, such as skeletal muscle-specific transcription factor MyoD or skeletal muscle-specific myosin, or electrophysiological characteristics and morphologic characteristics such as fusion into a multinucleated striated fiber.

By “myocardium” is meant the muscular portion of the heart. The myocardium includes three major types of muscle fibers: atrial muscle fibers, ventricular muscle fibers, and specialized excitatory and conductive muscle fibers.

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a sequence of interest for gene therapy. Vectors include, for example, transposons and other site-specific mobile elements, viral vectors, e.g., adenovirus, adeno-associated virus (AAV), poxvirus, papillomavirus, lentivirus, herpesvirus, foamivirus and retrovirus vectors, and including pseudotyped viruses, liposomes and other lipid-containing complexes, and other macromolecular complexes capable of mediating delivery of a polynucleotide to a host cell, e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes. Vectors can also comprise other components or functionalities that further modulate gene delivery and/or gene expression, or that otherwise provide beneficial properties to the cells to which the vectors will be introduced. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described (see, e.g., Curiel et al., Proc. Natl. Acad. Sci. USA, 88:8850 (1991)).

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene may include a gene which is partly or entirely heterologous (i.e., foreign) to the transgenic organism, or may represent a gene homologous to an endogenous gene of the organism.

By “transgenic cell” is meant a cell containing a transgene. For example, a stem cell transformed with a vector containing an expression cassette can be used to produce a population of cells having altered phenotypic characteristics.

The term “transduction” denotes the delivery of a polynucleotide to a recipient cell either in vivo or in vitro, via a viral vector and preferably via a replication-defective viral vector, such as via a recombinant AAV.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” or “sequence” which “encodes” a particular protein, is a nucleic acid molecule which is transcribed and optionally also translated into a gene product, i.e., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

By “enhancer element” is meant a nucleic acid sequence that, when positioned proximate to a promoter, confers increased transcription activity relative to the transcription activity resulting from the promoter in the absence of the enhancer domain. Hence, an “enhancer” includes a polynucleotide sequence that enhances transcription of a gene or coding sequence to which it is operably linked. A large number of enhancers, from a variety of different sources are well known in the art. A number of polynucleotides which have promoter sequences (such as the commonly-used CMV promoter) also have enhancer sequences.

By “tissue-specific enhancer or promoter” is meant an element, which, when operably linked to a promoter or alone, respectively, directs gene expression in a particular cell type and does not direct gene expression in all tissues or all cell types. Tissue-specific enhancers or promoters may be naturally occurring or non-naturally occurring. One skilled in the art will recognize that the synthesis of non-naturally occurring enhancers or promoters can be performed using standard oligonucleotide synthesis techniques.

“Operably linked” refers to a juxtaposition, wherein the components so described are in a relationship permitting them to function in their intended manner. By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. Thus, a signal or targeting peptide sequence is operably linked to another protein if the resulting fusion is secreted from a cell as a result of the presence of a secretory signal peptide or into an organelle as a result of the presence of an organelle targeting peptide.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide or polypeptide refers to a nucleic acid sequence, peptide or polypeptide that is identified and separated from at least one contaminant nucleic acid, polypeptide or other biological component with which it is ordinarily associated in its natural source. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “peptide”, “polypeptide” and protein” are used interchangeably herein unless otherwise distinguished. These terms also include proteins that are post-translationally modified through reactions that include glycosylation, acetylation and phosphorylation.

By “growth factor” is meant an agent that, at least, promotes cell growth or induces phenotypic changes.

The term “angiogenic growth factor” means an agent that alone or in combination with other agents induces angiogenesis, and includes, but is not limited to, fibroblast growth factor (FGF), vascular endothelial growth factor (VEGF), hepatocyte growth factor, angiogenin, transforming growth factor (TGF), tissue necrosis factor (TNF, e.g., TNF-α), platelet derived growth factor (PDGF), granulocyte colony stimulatory factor (GCSF), placental GF, IL-8, proliferin, angiopoietin, e.g., angiopoietin-1 and angiopoietin-2, thrombospondin, ephrin-A1, E-selectin, leptin and heparin affinity regulatory peptide.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

A “cytokine” is a relatively low molecular weight protein secreted by cells, e.g., cells of the immune system, for the purpose of altering the function(s) of those cells and/or adjacent cells. Cytokines include interleukins, e.g., molecules which regulate the inflammatory and immune response, as well as growth and colony stimulating factors. By “growth factor” is meant an agent that, at least, promotes cell growth or induces phenotypic changes. Exemplary growth factors include, but are not limited to, fibroblast growth factor (FGF), vascular endothelial growth factor (VEGF), hepatocyte growth factor (HGF), transforming growth factor (TGF), platelet derived growth factor (PDGF), granulocyte colony stimulatory factor (G-CSF), placental GF, stem cell growth factor (SCF), or insulin-like growth factor (IGF).

A “drug” as used herein is an agent that is not a protein which is naturally produced by a cell or tissue, but which, in an effective amount, has a prophylactic or therapeutic effect.

General Overview

This document describes, among other things, method and apparatus for detecting physiological molecules in vivo. In one embodiment, tissue or cells capable of detecting one or more physiological molecules are administered (transplanted or implanted), e.g., by inserting or applying, appropriate cellular material (“donor tissue or cells”) to an animal, e.g., to a vessel or tissue or cells of an animal (“endogenous tissue or cells”). Prior to implantation, donor tissue or cells may be subjected to in vitro conditioning with one or more stimuli which preferably yields cells with a desirable phenotype. In one embodiment, the donor tissue or cells are present in and/or on a biocompatible matrix, e.g., a collagen-based matrix, or on the surface of an implantable device such as a lead, e.g., one having a biocompatible matrix applied thereto. In one embodiment, the transplanted donor tissue or cells are then monitored for alterations in membrane electrical potential, e.g., by coupling the donor tissue or cells to an implantable device which can detect those alterations and optionally also provide electrical stimulation with properly positioned electrodes. Several embodiments are presented below to provide examples of different apparatus and methods. It is understood that other apparatus and method are possible as provided by the attached claims and their equivalents.

Cell-based biosensors use living cells or tissues as transduction elements to detect or monitor physiological and functional information in an animal. The detection of cellular metabolism gives direct evidence of the activity of specific molecules such as receptors, e.g., by monitoring the binding of a ligand for the receptor, such as the natural ligand, or an antagonist or agonist of the natural ligand, and contains more information than binding measurements from antibody- or enzyme-based arrays which only show that a ligand binds to a receptor independent from metabolic activities. Such tissue-based biosensors include the complete protein pattern and/or set of enzymes (in contrast to sensor devices using, for example, isolated enzymes or DNA fragments) and cofactors, reflecting optimal and stable signaling pathways and metabolic activities, a common feature of all living systems. Moreover, as cultured, dissociated cells may respond in a different manner than cultured monolayers or more complex cellular arrays to a ligand for a specific receptor, those monolayer and complex arrays may be employed in vitro to determine whether ligand and/or drug administration to electrophysiologically active cells yields an altered electrical profile. Thus, these monolayers and complex arrays can be used to obtain information about the electrophysiological effects of ligands, toxins and/or drugs on cells in vitro and in vivo.

A biosensor of the present invention is made of tissue or cells capable of carrying out a physiological function, i.e., binding a ligand found in physiological fluid. In a preferred embodiment, the tissue or cells which make up a subject biosensor are excitable tissue or cells. Excitable tissues respond to parameters or signals within the body and allow the integration or transmission of these signals as a feature of their excitable properties. The function carried out by the excitable tissue or cells can then be used to monitor the parameters affecting the function.

The donor tissue or cells of the invention may be genetically engineered to expand the range and/or sensitivity of monitored parameters. By “genetically engineered,” it is meant that the tissue or cells are transgenic, i.e., the genetically engineered tissue or cells comprise one or more coding sequences for one or more gene products which is/are heterologous or foreign to the genome of the cell, is/are not normally expressed in the tissue or cells and/or is/are expressed at a different level in the transgenic tissue or cells. Thus, for example, by genetically engineering one or more cells to express a cell surface receptor, such genetically engineered cells, when placed in a subject, are useful for monitoring physiologic signals in the subject. The donor tissue or cells which make up a biosensor of the present invention may include cells which have been engineered to produce different proteins, in addition to a receptor for a ligand found as in physiological fluid, proteins including but not limited to coagulation factors, serotonin, growth factors, hormones, or other receptors.

Donor tissue or cells may be administered via any route including, but not limited to, intramuscular, subcutaneous, buccal, rectal, intravenous or intracoronary administration. The number of cells to be administered can vary. For example, from 10² to 10¹⁰, e.g., from 10³ to 10⁹, 10⁴ to 10⁸, or 10⁵ to 10⁷, cells can be administered. The amount of tissue or cells in a biosensor may vary depending on the height, weight, gender, age and condition of the recipient. Agents which may enhance angiogenesis may optionally be present in a biosensor of the invention or administered separately.

The biosensors of the present invention may be placed, inserted or implanted in various locations within an recipient. For example, a biosensor may be placed in a subcutaneous pocket anywhere on, the body. Other locations for implantation of a biosensor include for example, the vascular lumen. The heart of a biosensor recipient may also be the location for a biosensor of the invention. The biosensor when implanted alone or as part of a device is preferably implanted in a manner such that the donor tissue or cells are in direct communication with blood-borne substances delivered via the endogenous blood supply.

A biosensor recipient may be any animal. Preferably, the animal is a mammal. Examples of mammals which may be recipients of a subject biosensor include but are not limited to a mouse, rat, rabbit, pig, cat, dog, cattle, horse or sheep. Preferably, the mammal is a human.

Donor tissue or cells capable of carrying out a physiological function which can be used to monitor a physiological variable associated with the physiological function, are placed into a recipient, either implanted directly or as part of a system. Implantation may be performed on an animal recipient via a surgical procedure or by means of a catheter or tubing. Preferably, the recipient is placed under a general or local anesthesia during the implantation procedure. The donor tissue or cells may be connected to a device such as a delivery device or other implantable device. Examples of delivery devices include but are not limited to an electronic pacemaker, insulin pump, or drug pump. For example, donor tissue or cells may be placed on the tips of a catheter, tube or tubing which is then placed into an animal recipient. The device may also be a wire which is coated with donor tissue or cells and then put in contact with blood, such as when placed within an artery or vein. Preferably, the donor tissue or cells are excitable tissue or cells. In this aspect of the invention, the wire serves as a signaling device in detecting the level of a physiological molecule. Another example of a device which may form part of a biosensor are wire leads connected to a recipient heart, either through direct implantation of the biosensor in the recipient heart tissue or vasculature or indirectly through the placement of the wires of the biosensor device in the recipient heart tissue or vasculature. In still another example, the device may be an electronic pacemaker having a component which connects to the body. The component may house donor tissue or cells. The output from the device may be regulated in response to the physiological function of the donor tissue or cells.

Biosensors include donor tissue or cells and optionally a biocompatible material to which the donor tissue or cells are attached, embedded in and/or encapsulated in, which donor tissue or cells are optionally coupled to endogenous tissue or cells and/or optionally electrically coupled to an implantable medical device. The donor tissue or cells in a biosensor may be coupled via an electrical interface to an electronic measuring device or an electronic amplifying device. This embodiment is especially useful for monitoring physiological function. For example, a biosensor comprising donor tissue or cells may be coupled via an electrical interface to an electronic measuring device or an electronic amplifying device. Examples of electrical interfaces include but are not limited to silicon chips, magnetic field sensors, and field electrodes. Examples of electronic measuring devices include electrodes and field effect transistors. Examples of electronic amplifying devices include operational amplifier circuits.

Thus, the nature of a biosensor of the present invention can vary. A biosensor of the present invention may be placed in the body where it can communicate with monitoring devices either outside the body (external) or also within the body (internal). Such devices may be coupled to recording devices as well as to devices which deliver therapies such as drugs, electrical stimulations, or other agents or actions. The invention may be used to regulate the output of a signal, substance, or action from an implantable device or a delivery device. In this aspect of the invention, the monitoring function of the donor tissue or cells is integrated with an implantable device or delivery device to regulate the delivery of an electrical signal or drug, or a gene product expressed by a prokaryotic or eukaryotic cell, for example, a hormone such as insulin or a growth factor, to a recipient.

The present invention also provides a method for monitoring a physiological function. Tissue or cells capable of carrying out a physiological function, which can be used to monitor a physiological variable associated with the physiological function, are placed an animal recipient, either implanted directly or as part of a system. The physiological function of the donor tissue or cells is then monitored.

Donor Cells for Biosensors

Sources for donor cells include but are not limited to bone marrow-derived cells, e.g., mesenchymal cells and stromal cells, smooth muscle cells, fibroblasts, SP cells, pluripotent cells or totipotent cells, e.g., teratoma cells, hematopoietic stem cells, for instance, cells from cord blood and isolated CD34⁺ cells, adult stem cells, e.g., multipotent adult progenitor cells (MAPCs), embyronic stem cells, skeletal muscle derived cells, for instance, skeletal muscle cells and skeletal myoblasts, cardiac derived cells, myocytes, e.g., ventricular myocytes, atrial myocytes, SA nodal myocytes, AV nodal myocytes, and Purkinje cells. Subpopulations of adult stem cells have exhibited pluripotent characteristics, with the ability to differentiate into neuronal tissue (Jiang et al., Nature, 418:41 (2002)). In one embodiment, the donor cells are autologous cells, however, non-autologous cells, e.g., xenogeneic cells, may be employed. Donor cells may be isolated from cardiac tissue, skeletal muscle tissue, bone marrow or umbilical cord blood. These or similar cell populations may be capable of differentiation into electrically excitable cells, e.g., cardiac cells. Methods of culturing cells and/or methods of inducing differentiation of cells are known to the art. For example, methods to induce differentiation of ES cells, bone marrow cells, or hematopoietic stem cells to cardiac cells, are described in U.S. patent application Ser. No. 10/722,115, entitled “METHOD AND APPARATUS FOR CELL AND ELECTRICAL THERAPY OF LIVING TISSUE”.

In one embodiment, tissues or cells for use in the biosensors of the present invention are cardiac or neuronal tissue or cells. Cardiac tissue or cells and neuronal tissue or cells may be obtained from various sources such as donated organs or live donors. Heart tissue or cells and neuronal tissue or cells from either a donated organ or a live donor may be further cultured prior to use in a biosensor. Methods for culturing cardiac tissue or cells are well known and can be found in, for example, Rust et al. (Mol. Cell. Biochem., 181:143 (1998)). Methods for culturing neuronal tissue or cells are also well known and may be found in, for example, Barnea et al. (Res. Protoc., 4:156 (1999)). The donor tissue or cells and biosensor recipient should be as closely phylogenetically related as possible. For example, when the biosensor recipient is a human, cardiac tissue or cells and/or neuronal tissue or cells from a human or pig may be used. Preferably, human tissue or cells are used in a biosensor to be implanted in a human. Thus, a biosensor recipient may serve as tissue or cell donor. Alternatively, cardiac tissue or cells, or neuronal tissue or cells from a donor other than the biosensor recipient may be used. When cardiac or neuronal tissue or cells from a donor other than the biosensor recipient is used, the tissue or cells and the individual recipient are preferably HLA typed and matched.

The tissue or cells which make up a biosensor may be stem cell-derived cardiac myocytes. In this embodiment, stem cells are used to culture cardiac myocytes. Stem cells may be obtained from various sources such as, e.g., bone marrow, peripheral blood, organs, or tissue, including fat or umbilical cord blood, as well as any combination of these sources. The stem cells may be allogeneic (foreign to a biosensor recipient) or syngeneic (same to the biosensor recipient).

The donor cells can optionally be expanded in vitro to provide an expanded population of donor cells and/or to provide a two- or three-dimensional structure, optionally in combination with a biocompatible material, e.g., matrix. For instance, donor cells may be treated in vitro by subjecting them to mechanical, electrical, or biological conditioning, or any combination thereof, as described in U.S. patent application Ser. No. 10/722,115, entitled “METHOD AND APPARATUS FOR CELL AND ELECTRICAL THERAPY OF LIVING TISSUE”, which is incorporated by reference herein. The conditioning may include continuous or intermittent exposure to exogenous stimuli. Mechanical conditioning includes subjecting donor cells to a mechanical stress that simulates the mechanical forces applied upon cardiac muscle cells in the myocardium due to the cyclical changes in heart volume and blood pressure. Electrical conditioning includes subjecting donor cells to electrical conditions that simulate the electrical conditions in the myocardium which result in contraction of the heart. Biological conditioning includes subjecting donor cells to exogenous agents, e.g., differentiation factors, growth factors, angiogenic proteins, survival factors, and cytokines, as well as to expression cassettes (transgenes) optionally in addition to the expression cassette encoding the protein capable of associating with a cell membrane and binding a ligand, for instance, expression cassettes encoding a gene product including, but not limited to, an angiogenic protein, a growth factor, a differentiation factor, a survival factor, a cytokine, a cardiac cell-specific structural gene product, a cardiac cell-specific transcription factor, or a membrane protein, or comprising an antisense sequence, for instance, a ribozyme, or any combination thereof.

Expression Cassettes

The expression cassette optionally includes at least one transcription control element (transcription regulatory element) such as a promoter, optionally a regulatable promoter, e.g., one which is inducible or repressible, an enhancer, including a tissue- or cell-specific enhancer, or a transcription termination sequence. Preferably, the promoter and/or enhancer is one which is cell- or tissue-specific, e.g., cardiac cell-specific. For instance, the enhancer may be a muscle creatine kinase (mck) enhancer, and the promoter may be an alpha-myosin heavy chain (MyHC) or beta-MyHC promoter (see Palermo et al., Circ. Res., 78, 504 (1996)).

For purposes of the present invention, cell or tissue-specific control elements, such as neuronal- or muscle-specific and inducible promoters, enhancers and the like, will be of particular use. Such muscle-specific control elements include, but are not limited to, those derived from the actin and myosin gene families, such as from the myoD gene family (Weintraub et al., Science, 251, 761 (1991)); the myocyte-specific enhancer binding factor MEF-2 (Cserjesi and Olson, Mol. Cell Biol., 11, 4854 (1991)); control elements derived from the human skeletal actin gene (Muscat et al., Mol. Cell Bio., 7, 4089 (1987)) and the cardiac actin gene; muscle creatine kinase sequence elements (Johnson et al., Mol. Cell Biol., 9, 3393 (1989)) and the murine creatine kinase enhancer (mCK) element; control elements derived from the skeletal fast-twitch troponin C gene, the slow-twitch cardiac troponin C gene and the slow-twitch troponin I gene; hypoxia-inducible nuclear factors (Semenza et al., Proc. Natl. Acad. Sci. USA, 88, 5680 (1991); Semenza et al., J. Biol. Chem., 269, 23757); steroid-inducible elements and promoters, such as the glucocorticoid response element (GRE) (Mader and White, Proc. Natl. Acad. Sci. USA, 90, 5603 (1993)); the fusion consensus element for RU486 induction; and elements that provide for tetracycline regulated gene expression (Dhawan et al., Somat. Cell. Mol. Genet., 21, 233 (1995); Shockett et al., Proc. Natl. Acad. Sci. USA, 92, 6522 (1995)).

Cardiac cell restricted promoters include but are not limited to promoters from the following genes: a α-myosin heavy chain gene, e.g., a ventricular α-myosin heavy chain gene, β-myosin heavy chain gene, e.g., a ventricular ≢-myosin heavy chain gene, myosin light chain 2v gene, e.g., a ventricular myosin light chain 2 gene, myosin light chain 2a gene, e.g., a ventricular myosin light chain 2 gene, cardiomyocyte-restricted cardiac ankyrin repeat protein (CARP) gene, cardiac α-actin gene, cardiac m2 muscarinic acetylcholine gene, ANP gene, BNP gene, cardiac troponin C gene, cardiac troponin I gene, cardiac troponin T gene, cardiac sarcoplasmic reticulum Ca-ATPase gene, skeletal α-actin gene, as well as an artificial cardiac cell-specific promoter.

Further, chamber-specific promoter promoters may also be employed, e.g., for atrial-specific expression, the quail slow myosin chain type 3 (MyHC3) or ANP promoter, may be employed. For ventricle-specific expression, the iroquois homeobox gene may be employed. Nevertheless, other promoters and/or enhancers which are not specific for cardiac cells or muscle cells, e.g., RSV promoter, may be employed in the expression cassettes and methods of the invention.

Other sources for promoters and/or enhancers are promoters and enhancers from the Csx/NKX 2.5 gene, titin gene, α-actinin gene, myomesin gene, M protein gene, cardiac troponin T gene, RyR2 gene, Cx40 gene, and Cx43 gene, as well as genes which bind Mef2, dHAND, GATA, CarG, E-box, Csx/NKX 2.5, or TGF-beta, or a combination thereof.

In other embodiments, disease-specific control elements may be employed. Nevertheless, other promoters and/or enhancers which are not specific for cardiac cells or muscle cells, e.g., RSV promoter, may be employed in the expression cassettes and methods of the invention. Other sources for promoters and/or enhancers are promoters and enhancers from the Csx/NKX 2.5 gene, titin gene, α-actinin gene, myomesin gene, M protein gene, cardiac troponin T gene, RyR2 gene, Cx40 gene, and Cx43 gene, as well as genes which bind Mef2, dHAND, GATA, CarG, E-box, Csx/NKX 2.5, or TGF-beta, or a combination thereof.

Delivery of exogenous transgenes may be accomplished by any means, e.g., transfection with naked DNA, e.g., a vector comprising the transgene, liposomes, calcium-mediated transformation, electroporation, or transduction, e.g., using recombinant viruses. A number of transfection techniques are generally known in the art. See, e.g., Graham et al., Virology, 52, 456 (1973), Sambrook et al., Molecular Cloning: A Laboratory Manual, Cold Spring Harbor Laboratories, New York (1989), Davis et al., Basic Methods in Molecular Biology, Elsevier (1986) and Chu et al., Gene, 13, 197 (1981). Particularly suitable transfection methods include calcium phosphate co-precipitation (Graham et al., Virol., 52, 456 (1973)), direct microinjection into cultured cells (Capecchi, Cell, 22, 479 (1980)), electroporation (Shigekawa et al., BioTechniques, 6, 742 (1988)), liposome-mediated gene transfer (Mannino et al., BioTechniques, 6, 682 (1988)), lipid-mediated transduction (Felgner et al., Proc. Natl. Acad. Sci. USA, 84, 7413 (1987)), and nucleic acid delivery using high-velocity microprojectiles (Klein et al., Nature, 327, 70 (1987)). Preferred recombinant viruses to deliver exogenous transgenes to cells include recombinant lentiviruses, retroviruses, adenoviruses, adeno-associated viruses (AAV), and herpes viruses including cytomegalovirus.

Gene Delivery Vectors

Gene delivery vectors include, for example, viral vectors, liposomes and other lipid-containing complexes, and other macromolecular complexes capable of mediating delivery of a gene to a host cell. Such vectors can be used to deliver genes in vitro or in vivo. In one embodiment, an expression cassette is introduced to donor tissue or cells in vitro. In another embodiment, an expression cassette is introduced to donor tissue or cells in vivo. In one embodiment, one expression cassette is introduced to donor tissue another expression cassette is introduced, e.g., intravenously or subcutaneously to the animal with the transplanted transgenic tissue or cells. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Gene delivery vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene vectors are described below.

Retroviral vectors exhibit several distinctive features including their ability to stably and precisely integrate into the host genome providing long-term transgene expression. Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes. Although lentiviruses have specific tropisms, pseudotyping the viral envelope with vesicular stomatitis virus yields virus with a broader range (Schnepp et al., Meth. Mol. Med., 69:427 (2002)).

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes.

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

Herpes simplex virus 1 (HSV-1) has a number of important characteristics that make it an important gene delivery vector. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has obviously strong tropism towards nerve cells. It has a very large genome size and can accommodate very large transgenes (>35 kb). Herpesvirus vectors are particularly useful for delivery of large genes.

Plasmid DNA is often referred to as “naked DNA” to indicate the absence of a more elaborate packaging system. Plasmid DNA may be delivered to cells as part of a macromolecular complex, e.g., a liposome or DNA-protein complex, and delivery may be enhanced using techniques including electroporation.

Biocompatible Materials for Biosensors

The biocompatible material, e.g., a biocompatible matrix, may be embedded and/or coated with donor tissue or cells and is optionally suitable for retaining and/or immobilizing donor tissue or cells or optionally other agents including other therapeutic agents under physiological conditions for a sustained period of time, e.g., for months or years once those cells are implanted. Once donor tissue or cells are embedded in or applied to a biocompatible material, it can be introduced to an animal or, prior to transplantation, coupled to an implantable medical device. Alternatively, the biocompatible material may be first coupled to the implantable medical device and then the donor tissue or cells embedded in or applied thereto.

Donor tissue or cells which optionally are embedded in or coated on a biocompatible matrix may be enclosed in a semipermeable membrane which allows the transport of low molecular weight substances inward and outward, permitting cell survival and function, and prevent entry and exit of large molecules, e.g., entry of undesirable molecules such as antibodies and immune cells, and the exit of the donor tissue or cells. For instance, microcapsules and hollow fibers with a semipermeable wall may be employed. Molecular weight cut offs for the semipermeable membrane may be about 50 to 100 kD. The membrane may be made of any suitable material which is nondegradable and biocompatible, e.g., agarose, polyvinyl alcohol, e.g., cross-linked polyvinyl alcohol, polyacrylates, polyamides, and polyurethane, and including a dialysis membrane, nylon or polysultoxy, cellulose, e.g., cellulose acetate or methyl cellulose. The semipermeable materials may also be conjugated with heparin and/or polyethlyene glycol (PEG) to decrease immunogenic response, blood clotting and cell attachment on the surface. Examples of such enclosures and semipermeable membranes are discussed in U.S. Pat. No. 5,593,852; U.S. Pat. No. 5,431,160: U.S. Pat. No. 5,372,133; U.S. Pat. No. 4,919,141, and U.S. Pat. No. 4,703,756.

Biocompatible materials suitable for biocompatible matrices include polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof.

Additionally, it is possible to construct matrices from natural proteins or materials which may be crosslinked using a crosslinking agent such as 1-ethyl-3-(3-dimethylamino-propyl)carbodiimide hydrochloride. Such natural materials include albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or other “isolated materials”. An “isolated” material has been separated from at least one contaminant structure with which it is normally associated in its natural state such as in an organism or in an in vitro cultured cell population.

In one embodiment, the material may include liposomes, a hydrogel, cyclodextrins, nanocapsules or microspheres. Thus, a biocompatible material includes synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide (Roy et al., Mol. Ther., 7:401 (2003)), poly orthoesters (Heller et al., Adv. Drug Delivery Rev., 54:1015 (2002)), silk-elastin-like polymers (Megeld et al., Pharma. Res., 19:954 (2002)), alginate (Wee et al., Adv. Drug Deliv. Rev., 31:267 (1998)), EVAc (poly(ethylene-co-vinyl acetate), microspheres such as poly(D,L-lactide-co-glycolide) copolymer and poly(L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, the donor tissue or cells are embedded in or applied to a biocompatible material, e.g., a nonionic or ionic biodegradable or nonbiodegradable matrix, including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols.

In some embodiments, the biocompatible polymeric material is a biodegradable polymeric such as collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), poly(dioxanone) (PPS) or cellulose derivatives such as cellulose acetate. In an alternative embodiment, a biologically derived polymer, such as protein, collagen, e.g., hydroxylated collagen, or fibrin, or polylactic-polyglycolic acid or a polyanhydride, is a suitable polymeric matrix material.

In another embodiment, the biocompatible material includes polyethyleneterephalate, polytetrafluoroethylene, copolymer of polyethylene oxide and polypropylene oxide, a combination of polyglycolic acid and polyhydroxyalkanoate, or gelatin, alginate, collagen, hydrogels, poly-3-hydroxybutyrate, poly-4-hydroxybutyrate, and polyhydroxyoctanoate, and polyacrylonitrilepolyvinylchlorides.

For anchorage dependent cells which are attached to and/or embedded in (seeded on) biocompatible matrices the following polymers may be employed, e.g., natural polymers such as starch, chitin, glycosaminoglycans, e.g., hyaluronic acid, dermatan sulfate and chrondrotin sulfate, collagen, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone. The incorporation of molecules such as tricalciumphosphate, hydroxyapetite and basic salts into a polymer matrix can alter the degradation and resorption kinetics of the matrix. Moreover, the properties of polymers can be modified using cross-linking agents.

In one embodiment, the biocompatible material is isolated ECM. ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like. The preparation and use of isolated ECM in vivo is described in co-pending, commonly assigned U.S. patent application Ser. No. Ser. No. 11/017,237, entitled “USE OF EXTRACELLULAR MATRIX AND ELECTRICAL THERAPY,” filed on Dec. 20, 2004, which is hereby incorporated by reference in its entirety.

In one embodiment, ECM is isolated from the small intestine. Intestinal submucosal tissue for use in the invention typically comprises the tunica submucosa delaminated from both the tunica muscularis and at least the luminal portions of the tunica mucosa. In one embodiment, the submucosal tissue comprises the tunica submucosa and basilar portions of the tunica mucosa including the lamina muscularis mucosa and the stratum compactum. The preparation of submucosal tissue is described in U.S. Pat. No. 4,902,508 and Bell, In: Tissue Engineering: Current Perspectives, Cambridge, Mass., Burkhauser Publishers, pp. 179-189 (1993), the disclosures of which are expressly incorporated herein by reference. For example, a segment of vertebrate intestine, preferably harvested from porcine, ovine or bovine species, or other warn blooded vertebrates, is rinsed free of contents, then split longitudinally to form a sheet and delaminated. In particular, the superficial layers of the tunica mucosa are removed by mechanical delamination. The tissue is then turned to the opposite side and the tunica muscularis externa and tunica serosa are mechanically removed leaving the tunica submucosa and the basilar layers of the tunica mucosa. The remaining tissue represents isolated ECM and may include a small number of intact cells.

In one embodiment, ECM is isolated from the urinary bladder. The wall of the urinary bladder is composed of the following layers: the mucosa (including a transitional epithelium layer and the tunica propria), a submucosa layer, up to three layers of muscle and the adventitia (a loose connective tissue layer)—listed in cross-section from luminal to abluminal sides. Urinary bladder submucosa may be prepared from bladder tissue harvested from animals raised for meat production, including, for example, porcine, ovine or bovine species or other warm-blooded vertebrates. For example, the urinary bladder is harvested and thoroughly rinsed in tap water to remove its contents. The bladder is split open through the apex and bisected to yield roughly equal-sized halves that are prepared separately. The luminal side of the bladder is placed face down and the external muscle layers, i.e., muscularis externa (smooth muscle cell layers and serosa), are removed by mechanical delamination. The transitional epithelium of the urinary bladder is removed by either mechanical or ionic methods (e.g., 1.0 N NaCl treatment) leaving behind tissue corresponding to isolated ECM, e.g., approximately a 50 μM to 80 μM thick sheet of ECM that originally resides between the transitional epithelium and the smooth muscle layers of the urinary bladder, i.e., the submucosa and basement membrane of the transitional epithelium.

In another embodiment, ECM from bladder wall segments or small intestine is prepared using a modification to the technique in Meezan et al. (Life Sci., 17:1721 (1975)). The method in Meezan et al. includes placing tissue fractions in a large volume (100:1) of distilled water containing 0.1% sodium azide and magnetically stirring the mixture for 1-2 hours in order to lyse the cells and release the intracellular contents. The lysed tissue suspension is then centrifuged to yield a firm pellet, and the supernatant discarded. The pellet is suspended in 40 ml of 1M NaCl and 2000 Kunitz units of DNAase (Sigma, Deoxyribonuclease 1) are added and the suspension stirred for 1-2 hours. The mixture is again centrifuged to bring down a firm pellet and the supernatant discarded. The pellet is then suspended in 40 ml of 4% sodium deoxycholate containing 0.1% sodium azide and stirred for 2-4 hours at room temperature. The mixture is centrifuged, the supernatant discarded, and the pellet either washed several times with water by centrifugation and resuspension, or by extensive irrigation on a 44 micron nylon sieve (Kressilk Products, Inc., Monterey Park, Calif.). In the modified method, the time of incubation with sodium deoxycholate and sodium azide is increased and additional washing procedures incorporated. Accordingly, first, the mucosa is scraped off mechanically. Afterwards all cell structures of the remaining tissue are eliminated chemically and enzymatically leaving the acellularized muscularis layer. This is achieved with subsequent exposure to a hypoosmolar and hyperosmolar solutions of crystalloids. In addition, a final treatment with sodium deoxycholate destroys remaining cell structures. After following washing procedures, the resulting material, which provides cross-linked fibres of the submucosa with the remaining muscularis collagen-elastin framework, can be stored in PBS solution, e.g., with antibiotics at 4° C. for a few months.

Isolated ECM can be cut, rolled, or folded.

Fluidized forms of submucosal tissue are prepared by comminuting submucosa tissue by tearing, cutting, grinding, or shearing the harvested submucosal tissue. Thus, pieces of submucosal tissue can be comminuted by shearing in a high speed blender, or by grinding the submucosa in a frozen or freeze-dried state, to produce a powder that can thereafter be hydrated with water or buffered saline to form a submucosal fluid of liquid, gel or paste-like consistency.

The comminuted submucosa formulation can further be treated with an enzymatic composition to provide a homogenous solution of partially solubilized submucosa. The enzymatic composition may comprise one or more enzymes that are capable of breaking the covalent bonds of the structural components of the submucosal tissue. For example, the comminuted submucosal tissue can be treated with a collagenase, glycosaminoglycanase, or a protease, such as trypsin or pepsin at an acidic pH, for a period of time sufficient to solubilize all or a portion of the submucosal tissue protein components. After treating the comminuted submucosa formulation with the enzymatic composition, the tissue is optionally filtered to provide a homogenous solution. The viscosity of fluidized submucosa for use in accordance with this invention can be manipulated by controlling the concentration of the submucosa component and the degree of hydration. The viscosity can be adjusted to a range of about 2 to about 300,000 cps at 25° C. Higher viscosity formulations, for example, gels, can be prepared from the submucosa digest solutions by adjusting the pH of such solutions to about 6.0 to about 7.0.

The present invention also contemplates the use of powder forms of submucosal tissues. In one embodiment, a powder form of submucosal tissue is prepared by pulverizing intestinal submucosa tissue under liquid nitrogen to produce particles ranging in size from 0.01 to 1 mm² in their largest dimension. The particulate composition is then lyophilized overnight, pulverized again and optionally sterilized to form a substantially anhydrous particulate composite. Alternatively, a powder form of submucosal tissue can be formed from fluidized submucosal tissue by drying the suspensions or solutions of comminuted submucosal tissue.

Submucosal tissue may be “conditioned” to alter the viscoelastic properties of the submucosal tissue. Submucosal tissue is conditioned by stretching, chemically treating, enzymatically treating or exposing the tissue to other environmental factors. The conditioning of submucosal tissue is described in U.S. Pat. No. 5,275,826, the disclosure of which is expressly incorporated herein by reference. In accordance with one embodiment, vertebrate derived submucosal tissues are conditioned to a strain of no more than about 20%.

In one embodiment, the submucosal tissue is conditioned by stretching the tissue longitudinally. One method of “conditioning” the tissue by stretching involves application of a given load to the submucosa for three to five cycles. Each cycle consists of applying a load to the tissue for five seconds, followed by a ten second relaxation phase. Three to five cycles produces a stretch-conditioned material. For example, submucosal tissue can be conditioned by suspending a weight from the tissue, for a period of time sufficient to allow about 10 to 20% or more elongation of the tissue segment. Optionally, the material can be preconditioned by stretching in the lateral dimension.

In one embodiment the submucosal tissue is stretched using 50% of the predicted ultimate load. The “ultimate load” is the maximum load that can be applied to the submucosal tissue without resulting in failure of the tissue (i.e., the break point of the tissue). Ultimate load can be predicted for a given strip of submucosal tissue based on the source and thickness of the material. Accordingly, one method of “conditioning” the tissue by stretching involves application of 50% of the predicted ultimate load to the submucosa for three to ten cycles. Each cycle consists of applying a load to the material for five seconds, followed by a ten second relaxation phase. The resulting conditioned submucosal tissue has a strain of less than 30%, more typically a strain from about 20% to about 28%. In one embodiment, conditioned the submucosal tissue has a strain of no more than 20%. The term strain as used herein refers to the maximum amount of tissue elongation before failure of the tissue, when the tissue is stretched under an applied load. Strain is expressed as a percentage of the length of the tissue before loading.

Typically the conditioned submucosal tissue is immobilized by clamping, suturing, stapling, gluing (or other tissue immobilizing techniques) the tissue to the support, wherein the tissue is held at its preconditioned length in at least one dimension. In one embodiment, delaminated submucosa is conditioned to have a width and length longer than the original delaminated tissue and the conditioned length and width of the tissue is maintained by immobilizing the submucosa on a support. The support-held conditioned submucosal tissue can be sterilized before or after being packaged.

Preferably, isolated ECM is decellularized, and optionally sterilized, prior to storage and/or use. In one embodiment, isolated ECM has a thickness of about 50 to 250 micrometers, e.g., 100 to 200 micrometers, and is >98% acellular. Numerous methods may be used to decellularize isolated ECM (see, for example, Courtman et al., J. Biomed. Materi. Res., 18:655 (1994); Curtil et al., Cryobiology, 34:13 (1997); Livesey et al., Workshop on Prosthetic Heart Valves, Georgia Inst. Tech. (1998); Bader et al., Eur. J. Cardiothorac. Surg., 14:279 (1998)). For instance, treatment of isolated ECM with dilute (0.1%) peracetic acid and rinsing with buffered saline (pH 7.0 to 7.4) and deionized water renders the material acellular with a neutral pH. Alternatively, isolated ECM is thoroughly rinsed under running water to lyse the remaining resident cells, disinfected using 0.1% peracetic acid in ethanol, and rinsed in phosphate buffered saline (PBS, pH=7.4) and distilled water to return its pH to approximately 7.0. Decellularization may be ascertained by hematoxylin-eosin staining.

Isolated, and optionally decellularized, ECM contains a mixture of structural and functional molecules such as collagen type I, III, IV, V, VI; proteoglycans; glycoproteins; glycosaminoglycans; and growth factors in their native 3-dimensional microarchitecture, including proteins that influence cell attachment, gene expression patterns, and the differentiation of cells. Isolated ECM is optionally sterilized and may be stored in a hydrated or dehydrated state.

Isolated ECM may be sterilized using conventional sterilization techniques including tanning with glutaraldehyde, formaldehyde tanning at acidic pH, ethylene oxide treatment, propylene oxide treatment, gas plasma sterilization, gamma radiation, electric beam radiation and peracetic acid sterilization. Sterilization techniques which do not adversely affect the mechanical strength, structure, and biotropic properties of the isolated ECM are preferred. For instance, strong gamma radiation may cause loss of strength of sheets of submucosal tissue. Preferred sterilization techniques include exposing isolated ECM to peracetic acid, low dose gamma irradiation, e.g., 1-4 mRads gamma irradiation or more preferably 1-2.5 mRads of gamma irradiation, or gas plasma sterilization. In one embodiment, peracetic acid treatment is typically conducted at a pH of about 2 to about 5 in an aqueous ethanolic solution (about 2 to about 10% ethanol by volume) at a peracid concentration of about 0.03 to about 0.5% by volume. After isolated ECM is sterilized, it may be wrapped in a porous plastic wrap or foil and sterilized again, e.g., using electron beam or gamma irradiation sterilization techniques. Isolated ECM for implantation is generally subjected to two or more sterilization processes. Terminal sterilization, e.g., with 2.5 mRad (10 kGy) gamma irradiation results in a sterile, pyrogen-free biomaterial. Isolated ECM or isolated, decellularized ECM may then be stored, e.g., at 4° C., until use. Lyophilized or air dried ECM can be rehydrated and used in accordance with this invention without significant loss of its properties. Decellularized and/or sterilized isolated ECM is substantially nonimmunogenic and has high tensile strength. Isolated ECM may, upon implantation, undergo remodeling (resorption and replacement with autogenous differentiated tissue), serve as a rapidly vascularized matrix for support and growth of new tissue, and assume the characterizing features of the tissue(s) with which it is associated at the site of implantation, which may include functional tissue.

In some embodiments, isolated ECM may be subjected to chemical and non-chemical means of cross-linking to modify the physical, mechanical or immunogenic properties of naturally derived ECM (Bellamkondra et al., J. Biomed. Mater. Res., 29:633 (1995)). Chemical cross-linking methods generally involve aldehyde or carbodiimide. Photochemical means of protein cross-linking may also be employed (Bouhadir et al., Ann. NY Acad. Sci., 842:188 (1998)). Cross-linking generally results in a relatively inert bioscaffold material which may induce a fibrous connective tissue response by the host to the scaffold material, inhibit scaffold degradation, and/or inhibit cellular infiltration into the scaffold. ECM scaffolds that are not cross-linked tend to be rapidly resorbed in contrast nonresorbable cross-linked materials or synthetic scaffolds such as Dacron or polytetrafluoroethylene (Bell, Tissue Engin., 1: 163 (1995); Bell, In: Tissue Engineering: Current Perspectives, Burhauser Pub. pp. 179-189 (1993); Badylak et al., Tissue Engineering, 4:379 (1998); Gleeson et al., J. Urol., 148:1377 (1992)).

Seeding of biocompatible materials with agents including drugs, cytokines, cells and/or vectors can be performed prior to and/or at the time of implantation. In one embodiment, seeding of biocompatible materials can be performed in a static two-dimensional chamber system or a three-dimensional rotating bioreactor. For instance, wet isolated ECM (2×3 cm in size) or tubular segments to be seeded are placed on the bottom of a chamber and covered with a liquid medium such as an aqueous medium, e.g., a cell culture medium, or perfused with such medium, for instance, over a period of up to 6 weeks in the presence of the one or more agents. Initially, for cell seeded matrices, approximately 1×10⁶ cells may be added to matrices. Additional cells may be added during subsequent culture. Cells may attach to matrices, e.g., to isolated ECM via several attachment proteins present within matrices, including type I collagen, type IV collagen, and fibronectin (Hodde et al., Tissue Engineering, 8:225 (2002)). Cells may grow to single-layer confluence on both surfaces of the matrix.

The above examples are provided for reference only, and the range of suitable materials should not be construed as limited to those materials listed above. In one embodiment, the donor tissue or cells form a two or three-dimensional shape which is covered with a semipermeable membrane to retain the donor tissue or cells therein.

Device

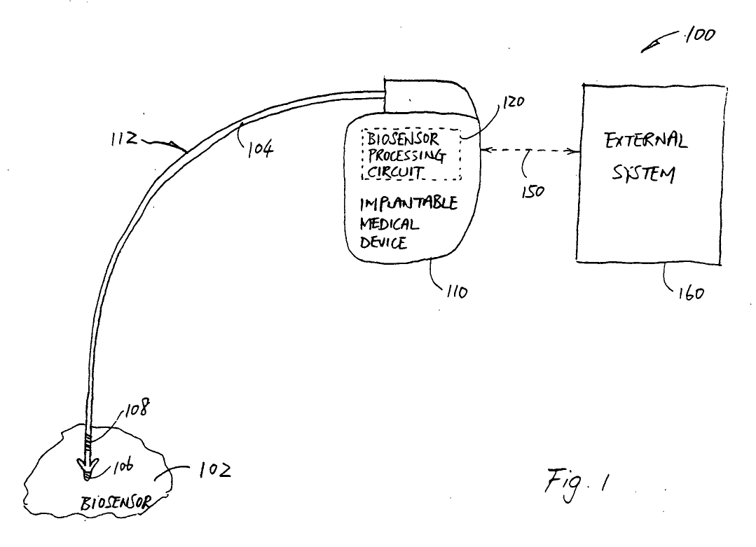

FIG. 1 is an illustration of an exemplary embodiment of a cardiac rhythm management (CRM) system 100 and portions of the environment in which CRM system 100 operates. CRM system 100 includes an implantable medical device 110 that is electrically coupled to a biosensor 102 via a lead system 112. An external system 160 communicates with implantable medical device 110 via a telemetry link 150.

Biosensor 102 includes donor tissue or cells capable of carrying out a physiological function that can be used to monitor a physiological variable associated with the physiological function. In one embodiment, biosensor 102 includes transgenic mammalian cells that are electrically excitable or are capable of differentiating into electrically excitable cells. The transgenic mammalian tissue or cells are augmented with an expression cassette. The expression cassette includes a transcriptional regulatory element operably linked to an open reading frame encoding a protein that is capable of associating with the cell membrane and binding a molecule found in physiological fluid of a mammal. The binding alters the amount and/or activity of one or more intracellular second messenger molecules. The one or more intracellular second messenger molecules in turn modulate the activity of one or more ion channels.

Implantable medical device 110 includes a hermetically sealed can housing a biosensor processing circuit 120 that is electrically coupled to biosensor 102 to monitor the physiological variable. In one embodiment, implantable medical device 110 is a biosensor processing device for monitoring one or more physiological variables and/or detecting one or more physiological events through biosensor 102. In other embodiments, in addition to biosensor processing circuit 120, implantable medical device 110 includes, but is not limited to, one or more of a pacemaker circuit, a cardioversion circuit, a defibrillation circuit, a cardiac resynchronization device, a cardiac remodeling control device, a neurostimulation circuit, a drug delivery device, a cell therapy device, a gene therapy device, and a monitoring circuit sensing other physiological variables or events. Implantable medical device 110 is also known as an implantable pulse generator or implantable generator when its functions include delivering of electrical stimulation pulses such as pacing, cardioversion, defibrillation, and neurostimulation pulses.

Lead system 112 includes one or more leads with electrodes to provide one or more electrical connections between implantable medical device 110 and biosensor 102. In one embodiment, as illustrated in FIG. 1, lead system 112 includes a bipolar sensing-pacing lead 104. Lead 104 includes a tip electrode 106 and a ring electrode 108. In one embodiment, lead 104 is connected to biosensor 102 such that electrodes 106 and 108 are in contact with the donor tissue or cells forming biosensor 102. In another embodiment, biological materials forming biosensor 102 are coated onto one or both of electrodes 106 and 108. In another embodiment, biological materials forming biosensor 102 are coated onto a portion of the elongate body of lead 104. In other embodiments, lead system 112 includes additional one or more leads such as unipolar and/or bipolar sensing-pacing leads, defibrillation leads, and neurostimulation leads.

External system 160 allows for programming of implantable medical device 110 and receives signals acquired by implantable medical device 110. In one embodiment, external system 160 includes a programmer. In another embodiment, external system 160 is a patient management system including an external device in proximity of implantable medical device 110, a remote device in a relatively distant location, and a telecommunication network linking the external device and the remote device. The patient management system allows access to implantable medical device 110 from a remote location, for purposes such as monitoring patient status and adjusting therapies. In one embodiment, telemetry link 150 is an inductive telemetry link. In an alternative embodiment, telemetry link 150 is a far-field radio-frequency telemetry link. Telemetry link 150 provides for data transmission from implantable medical device 110 to external system 160. This may include, for example, transmitting real-time physiological data acquired by implantable medical device 110, extracting physiological data acquired by and stored in implantable medical device 110, extracting therapy history data stored in implantable medical device 110, and extracting data indicating an operational status of implantable medical device 100 (e.g., battery status and lead impedance). Telemetry link 150 also provides for data transmission from external system 160 to implantable medical device 110. This may include, for example, programming implantable medical device 110 to acquire physiological data, programming implantable medical device 110 to perform at least one self-diagnostic test (such as for a device operational status), and programming implantable medical device 110 to deliver one or more therapies.

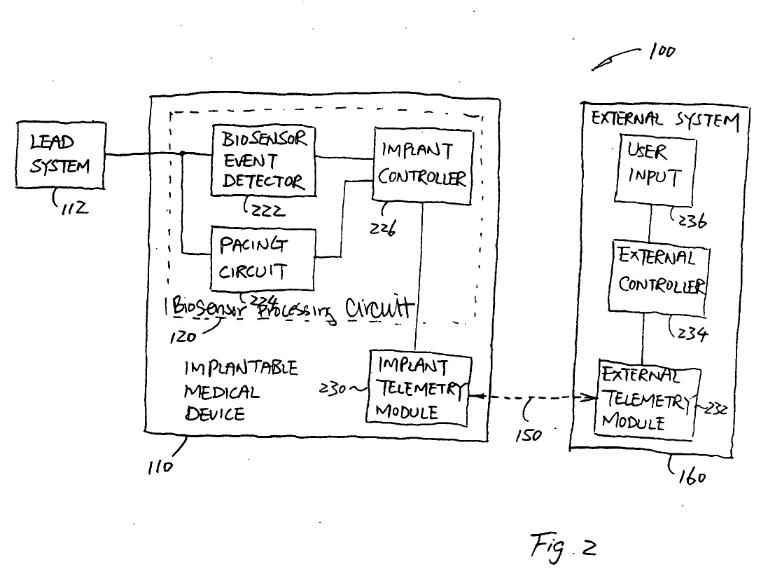

FIG. 2 is a block diagram illustrating an exemplary embodiment of portions of a circuit of CRM system 100. The circuit allows for the sensing of one or more physiological variables using biosensor 102. In addition to the system components illustrated in FIG. 2, CRM system 100 may include circuits performing one or more therapeutic functions and/or one or more additional sensing functions.

Implantable medical device 110 includes biosensor processing circuit 120 and an implant telemetry module 230. Biosensor processing circuit 120 includes a biosensor event detector 222, a pacing circuit 224, and an implant controller 226. Biosensor event detector 222 and pacing circuit 224 are both electrically coupled to biosensor 102 via lead system 112. Pacing circuit 224 delivers pacing pulses. Biosensor event detector 222 detects one or more physiological variables and/or events, including those affected or induced by the pacing pulses. Implant controller 226 includes a pacing control module to control the delivery of the pacing pulses and a signal processing module to produce signals representative of detected one or more physiological variables and/or events. In one embodiment, biosensor event detector 222 detects modulation in conduction and/or refractoriness, such as measured by the effective refractory period (ERP), in transgenic mammalian tissue or cells that form biosensor 102. Pacing circuit 224 delivers pacing pulses to the transgenic mammalian tissue or cells at predetermined pacing intervals. Biosensor event detector 222 includes an evoked response detector to detect tissue responses evoked by the pacing pulses. A tissue refractory period is measured or estimated based on the pacing intervals and number of successive pacing pulses that fail to evoke a tissue response. For instance, a ERP may be measured by determining the minimum stimulation threshold to elicit propagated activity in the tissue. An energy above the threshold (e.g., with a stimulation voltage at about twice a threshold voltage) is then used. A stimulation interval is chosen for the driver train of electrical pulses, e.g., an interval of 300 to 400 ms between pulses. Drive train pulses (S1), e.g., eight S1 pulses, are delivered. Then, a single pulse (S2) is delivered after a S1-S2 coupling interval, which is shorter than the S1 drive interval. This sequence may be repeated one or more times with different S1-S2 coupling intervals. S2 may elicit propagated activity. The longest S1-S2 interval that does not result in S2 propagating activity is the ERP.

External system 160 includes an external telemetry module 232, an external controller 234, and a user input 236. Implant telemetry module 230 and external telemetry module 232 supports telemetry 150 to perform bi-directional communications that includes transmitting any physiological variables and events from implantable medical device 110 to external system 160. External controller 234 controls the operation of external system 160. User input 236 allows a user such as a physician or other caregiver to control the operation of implantable medical device 110. In one embodiment, user input 236 allows the user to enter an external command for acquiring the ERP of the transgenic mammalian tissue or cells forming biosensor 102. The external command is transmitted to implantable medical device 110 through telemetry link 150, to be received by implant controller 226. In response, the pacing control module of implant controller 226 produces a signal for delivering pacing pulses, and the evoked response detector detects evoked responses for each delivery of a pacing pulse. Implant controller 226 then measures or estimates the ERP for the transgenic mammalian tissue or cells and produces a signal representative of the ERP. The signal is transmitted to external system 160 through telemetry link 150 to present to the user.

All publications, patents and patent applications referred to are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An implantable biosensor comprising: transgenic electrically excitable mammalian tissue or cells which are augmented with an expression cassette comprising a transcriptional regulatory element operably linked to an open reading frame encoding a protein which is capable of associating with the cell membrane and binding a molecule found in physiological fluid of a mammal, which binding alters the amount and/or activity of one or more intracellular second messenger molecules and which one or more intracellular second messenger molecules in turn modulate the activity of one or more ion channels, wherein the transgenic electrically excitable tissue or cells are optionally coupled via an electrical interface to an electronic measuring device or an electronic amplifying device.

2. The biosensor of claim 1 wherein the protein is a glucagon receptor, a receptor for a natriuretic peptide, an ion channel protein which binds ATP, an adrenergic receptor, a muscarinic receptor, an angiotensin receptor, or a vasopressin receptor.

3. The biosensor of claim 2 wherein the ion channel protein which binds ATP is an ATP-sensitive potassium channel protein.

4. The biosensor of claim 1 wherein the intracellular second messenger molecule is cAMP, cGMP, Ca²⁺, inositol triphosphate or diacylglycerol.

5. The biosensor of claim 1 wherein the transgenic electrically excitable mammalian tissue or cells further comprise a second expression cassette comprising a second transcriptional regulatory element operably linked to a second open reading frame encoding an ion channel protein.

6. The biosensor of claim 5 wherein the second expression cassette encodes a chloride channel protein or a nonselective cation channel protein.

7. The biosensor of claim 5 wherein the second transcriptional regulatory element is a promoter which is expressed in muscle cells.

8. The biosensor of claim 5 wherein the second open reading frame encodes an ion channel is sensitive to cAMP or cGMP.

9. The biosensor of claim 4 wherein the protein is a glucagon receptor and the binding of glucagon to the receptor increases the amount and/or activity of cAMP and/or cGMP.

10. The biosensor of claim 1 wherein the one or more ion channels include those associated with L-type Ca²⁺ current, Kᵣ⁺ current, fast Na⁺ current and/or Cl⁻ current.

11. The biosensor donor of claim 1 wherein the transcriptional regulatory element is a promoter which is expressed in muscle cells.

12. The biosensor of claim 1 wherein the excitable tissue or cells are cardiac tissue or cells.

13. The biosensor of claim 1 wherein the transgenic mammalian tissue or cells are incorporated in an implantable device and/or a biocompatible matrix.

14. The biosensor of claim 13 wherein the device is a patch, tube, tubing, catheter, stent, wire, defibrillator, implantable drug infusion pump, wire leads, or pacemaker.

15. The biosensor of claim 1 which further comprises a drug delivery device.

16. A system, comprising:
transgenic mammalian cells which are electrically excitable or are capable of differentiating into electrically excitable cells, wherein the transgenic mammalian cells are augmented with an expression cassette comprising a transcriptional regulatory element operably linked to an open reading frame encoding a protein which is capable of associating with the cell membrane and binding a molecule found in physiological fluid of a mammal, which binding alters the amount and/or activity of one or more intracellular second messenger molecules and which one or more intracellular second messenger molecules in turn modulate the activity of one or more ion channels; and
an implantable medical device including:
an event detector electrically coupled to the transgenic mammalian cells and adapted to detect a modulation in conduction and/or refractoriness in the transgenic mammalian cells as a result of the binding of the molecule to the protein; and
an implant controller coupled to the event detector, the implant controller adapted to produce a signal in response to the modulation in the conduction and/or refractoriness detected by the event detector.

17. The system of claim 16 further comprising at least one lead coupled between the event detector and the transgenic mammalian cells, the at least one lead including one or more electrodes configured for placement in or on the transgenic mammalian cells.

18. The system of claim 17 wherein the implantable medical device further comprises a pacing circuit coupled to the implant controller, and wherein the implant controller includes a pacing control module adapted to control a delivery of pacing pulses.

19. The system of claim 18 wherein the implantable medical device further comprises an implant telemetry module to receive an external command, and wherein the pacing control module is adapted to produce an electrical signal in response to the external command.

20. The system of claim 19 further comprising an external system communicatively coupled to the implantable medical device, the external system including:
a user input device to produce the external command; and
an external telemetry module to transmit the external command to the implant telemetry module.

21. The system of claim 20 wherein the external system comprises a programmer.

22. The system of claim 21 wherein the external system comprises an advanced patient management system including:
an external device wirelessly coupled to the implantable medical device via telemetry;
a remote device to provide for access to the implantable medical device from a distant location; and
a network connecting the external device and the remote device.

23. The system of claim 22 wherein the external device comprises the user input.

24. The system of claim 22 wherein the remote device comprises the user input.

25. The system of claim 17 wherein the implantable medical device further comprises an implant telemetry module coupled to the implant controller and adapted to transmit the signal to an external system.

26. The system of claim 25 wherein the external system includes a user input device to receive the transmitted signal.

27. The system of claim 17 wherein a biocompatible matrix comprises the transgenic mammalian tissue or cells.

28. The system of claim 17 wherein the signal is an electrical signal.

29. The system of claim 17 further comprising a drug delivery device.

30. The system of claim 17 wherein the device is a patch, tube, tubing, catheter, stent, wire, defibrillator, implantable drug infusion pump, wire leads, or pacemaker.

31. A method comprising monitoring in a mammal having the system of claim 16 a signal produced by the implant controller.

32. A method of regulating delivery of an electrical signal in a mammal from an external system comprising: monitoring the presence and/or amount of a molecule found in physiological fluid of a mammal having the system of claim 19, and regulating the electrical signal from the pacing control module in response to a modulation in the conduction and/or refractoriness of the transgenic electrically excitable mammalian tissue or cells.

33. The method of claim 32 wherein the transgenic electrically excitable tissue or cells are cardiac tissue or cells.

34. The method of claim 32 wherein the transgenic electrically excitable mammalian tissue or cells are incorporated in a biocompatible matrix.

35. The method of claim 32 wherein the implantable medical device further comprises a drug delivery device.

36. The system of claim 16 wherein the system comprises a tissue comprising the transgenic mammalian cells.
